(19) United States
(12) Patent Application Publication
(10) Pub. No.: US 2014/0249391 A1
(43) Pub. Date: Sep. 4, 2014

(54) TISSUE VISUALIZATION FOR RESECTION

(71) Applicant: Christopher J. Rothfuss, (US)

(72) Inventor: Sung-Wei Chen, Las Vegas, NV (US)

(73) Assignee: EMPIRE TECHNOLOGY DEVELOPMENT LLC, Wilmington, DE (US)

(21) Appl. No.: 13/879,489

(22) PCT Filed: Apr. 23, 2012
(86) PCT No.: PCT/US2012/034672

§ 371 Date: Sep. 10, 2013

(51) Int. Cl. A61K 49/00; A61M 5/00; A61B 5/00; A61K 49/04; A61B 17/22

(52) U.S. Cl. CPC A61K 49/0052; A61K 49/04; A61B 17/22; A61B 5/0071; A61M 5/007
USPC 600/317; 424/9.6; 424/9.42; 606/110; 600/431

(57) ABSTRACT

Methods for distinguishing between two interspersed biological tissues, for procedures such as surgical resection, include exposing the tissues to at least two components, a first of which components produces or is capable of producing a detectable signal, and the other of which components either blocks the produced signal of the first component or activates the first component to produce the detectable signal. One of the components is selectively taken up by one of the tissues at a concentration which is greater than the concentration by which it is taken up by the other tissue to provide a distinguishable difference in the detectable signal originating from the two tissues.

### BACKGROUND

Cancer, also medically referred to as malignant neoplasm, generally refers to one of a group of more than 100 diseases that are caused by the uncontrolled growth and spread of cells. Normal, non-cancerous cells, typically reproduce until maturation is attained and then only reproduce as necessary to replace wounded cells. Gene damage can alter the cells, resulting in cancerous cells growing among the non-cancerous cells. Cancer can take the form of solid tumors, lymphomas and non-solid cancers such as leukemia.

Tumors generally are classified as either being malignant or benign. Cancer cells may grow and divide endlessly without differentiating to mature, functional cells, forming what are referred to as malignant tumors. These cancer cells may crowd out nearby non-cancerous cells inhibiting proper functioning of tissue structures, and may eventually invade nearby body parts. Some cancers may also spread to more distant body parts through the lymphatic system, or even the bloodstream. Benign tumors generally do not grow uncontrollably, do not invade neighboring tissues, and do not spread throughout the body.

Cancers are more easily treated and cured if they are discovered and treated prior to metastasis. The survival of a patient may generally be influenced by the stage at which the cancer is diagnosed. The stage, generally categorized as 1-4, is determined by the extent of disease, with stage 1 cancers being those that are small and not invading the surrounding tissues, while stage 4 cancers have established tumors in tissues other than the organ in which the cancer started. Once cancer cells metastasize, they may travel through the bloodstream or lymphatic system to other body parts, where the cells can begin multiplying and developing into new tumors.

Cancers can be detected in a number of ways, including the presence of certain signs and symptoms, screening tests, or medical imaging. Once detected, cancers may be treated with chemotherapy, radiation therapy, surgery, or any combination thereof. Patients who have cancer that has not spread beyond a local area, frequently may be cured by completely resecting the tumor via surgery. Prior to the resection surgery, various images of the tumor may be obtained, such as X-rays, CT scans, MRI scans or PET images. These images are able to provide guidance for the surgeon, but at the time of surgery, these images cannot be generated in real time to guide the surgeon to the tumor. As a result, the surgeon must use the unaided senses of sight and feel to determine the location and extent of the tumor.

Since resection typically relies on only the surgeon's unaided sight and feel, it is not uncommon that residual tumor will be left inside the patient. Studies have shown that patients with residual tumors are at greater risk of dying of the cancer than those that have the tumor completely resected. Because of this, these patients require further, often debilitating, costly therapy in an attempt to arrest and treat the cancer left in the patient at the time of surgery. It is therefore desirable to provide a simple and improved method for distinguishing between two different and interspersed tissues to enable medical personnel to attend to at least one of the two tissues as needed. For cancerous tissue, this may enable the surgeon to remove all of the cancerous cells from affected tissue.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, a method for distinguishing interspersed first and second biological tissues may include administering an effective amount of each of a first component and a second component to the interspersed tissues. The first component is detectable and taken-up by both of the first and second biological tissues. The second component masks detectability of the first component and is taken-up by one of the first and second biological tissues at a concentration which is greater than a concentration which is taken up by the other of the first and second biological tissues to mask detectability of the first component in the one of the first and second biological tissues to a greater extent than any masking of detectability of the first component in the other of the first and second biological tissues.

In an additional embodiment, a method for distinguishing interspersed first and second biological tissues may include administering an effective amount of each of a first component and a second component to the interspersed tissues. The first component is detectable upon activation by the second component. One of the first and second components is taken-up by both of the first and second biological tissues. The other of the first and second components is taken-up by only one of the first and second biological tissues.

In a further embodiment, a method for resection of tissue includes selecting a first component and a second component, wherein the first component is detectable and is taken-up by both the non-cancerous tissue and the cancerous tissue, and the second component masks detectability of the first component and is taken-up by one of the non-cancerous tissue and the cancerous tissue at a concentration which is greater than a concentration which is taken up by the other of the non-cancerous tissue and the cancerous tissue to enable the non-cancerous tissue and the cancerous tissue to be distinguishable from one another, administering to a patient an effective amount of each of the first component and the second component, and surgically removing the distinguishable cancerous tissue from the patient.

### BRIEF DESCRIPTION OF THE FIGURES

FIG. 1 depicts a method for distinguishing one biological tissue from another interspersed biological tissue according to an embodiment.

FIG. 2 depicts an alternate method for distinguishing between two interspersed tissues according to an embodiment.

FIG. 3 depicts an additional method for distinguishing between two interspersed tissues according to an embodiment.

FIG. 4 depicts a method for resecting of cancerous liver tissue from non-cancerous liver tissue according to an embodiment.

### DETAILED DESCRIPTION

Many medical conditions of organs and tissues may have one section of the organ or tissue which is healthy and another adjoining or interspersed section which is diseased. Alternatively, the tissues might be present in different diseased states. Or, as might be the case for cancers, one tissue type may be growing within another. In any of these medical states or numerous others, it may be a medical necessity for the health of the patient to remove the diseased or cancerous tissue. It may also be medically preferred to leave as much of the healthy tissue intact to provide at least some remaining organ function when the tissue is part of an organ with which the body cannot function properly if it were to be completely removed.

Segmental resection (segmentectomy) is a surgical procedure to remove part of an organ or gland. Resection may also be used to remove a tumor and some of the adjoining tissue around it. For malignant tumors in which the cancer cells are growing and dividing endlessly, it is usually necessary to remove the tumor before it grows too large and begins affecting the function of the tissue in which it is growing and/or adjoining tissues for which it is competing for nutrients and space.

Cancer can be considered to be the result of cells that uncontrollably grow without following a typical growth-death cycle. Normal cells in the body follow an orderly path of growth, division, and death. If this process breaks down and cell death does not readily occur, cancerous tumors may begin to form. Unlike regular cells, cancer cells usually do not experience programmatic death and instead continue to grow and divide. This may lead to a mass of abnormal cells that grows out of control.

Cells can experience uncontrolled growth if damage or mutations to the cell DNA occur, which may result in damage to the genes involved in cell division. Four key types of genes are responsible for the cell division process: oncogenes tell cells when to divide, tumor suppressor genes tell cells when not to divide, suicide genes control apoptosis and tell the cell to kill itself if something goes wrong, and DNA-repair genes instruct a cell to repair damaged DNA. A cancer may occur when a cell's gene mutations make the cell unable to correct DNA damage and unable to commit suicide. Similarly, cancer is a result of mutations that inhibit oncogene and tumor suppressor gene function, leading to uncontrollable cell growth.

Thus, even a single cancer cell will generally carry the mutation that caused the cancer to begin growing, and that single cell may be capable of dividing and growing and forming a new mass of cancer cells. It is therefore desirable to surgically remove every cancer cell if possible. In an attempt to ensure that all the cancer cells are removed, the surgeon may typically remove additional healthy tissue which is adjacent to the cancer cells. Since most bodily tissues and organs are required for proper functioning of the body, it may also be desirable that as much healthy tissue be left to ensure that sufficient organ function remains.

Some cancers, like liver cancers, generally can only be cured through resection or transplantation, with resection generally being the only realistic treatment. Complete removal of cancer cells in the liver can, however, pose many difficulties, because the cancer cells can be virtually indistinguishable from normal, healthy cells. Methods and systems for distinguishing diseased cells from healthy, un-diseased cells are therefore necessary to give the patient the best opportunity for recovery and normal bodily functions.

Some methods for distinguishing diseased cells from undiseased cells may rely on molecular differences between the cells. Cancer specific biomarkers are one type of system which may be used. However, biomarkers can be expensive and may not be specific for the cancer for which they are needed. Imaging systems, such as PET systems, generally provide sufficient differentiation between tumor cells and adjoining healthy cells. However, such systems cannot readily be adapted for real-time surgical applications because of challenges with real-time integration, visual field compatibility, and spatial resolution. Various fluorescent reporters have also been developed, but they are non-specific, expensive and difficult to use in practice. There are also significant FDA regulatory hurdles to be overcome with such fluorescent reporters. In view of the above, most surgeons still rely on visual appearance and feel for differentiating between tumor and normal tissue.

An alternative method for providing improved visualization to distinguish between two interspersed tissues may use different molecular entities and their binding specificities. One of the entities may provide a detectable signal, or be capable of being activated to produce a detectable signal, and an additional entity may be utilized in conjunction with the first entity to either block the detectable signal, or provide the activation needed to produce the detectable signal. In addition, at least one of the two entities may have a specificity for binding to essentially only one of the two tissues, while the other entity may have non-specific binding for both of the tissues. Further, it would be advantageous if both entities were already FDA approved for use in medical treatment, thereby providing an improved safety profile and a shortened, less-costly approval pathway.

At least six different distinct pairings of entities may be provided:
1) a first detectable entity taken up by both tissues and a second blocking entity taken up by the first tissue—allowing the second tissue to remain visually detectable.
2) a first detectable entity taken up by both tissues and a second blocking entity taken up by the second tissue—allowing the first tissue to remain visually detectable;
3) a first activatable entity taken up by both tissues and a second activating entity taken up by the first tissue—allowing the first tissue to become visually detectable;
4) a first activatable entity taken up by both tissues and a second activating entity taken up by the second tissue—allowing the second tissue to become visually detectable;
5) a first activatable entity taken up by the first tissue and a second activating entity taken up by both tissues—allowing the first tissue to become visually detectable;
6) a first activatable entity taken up by the second tissue and a second activating entity taken up by both tissues—allowing the second tissue to become visually detectable.

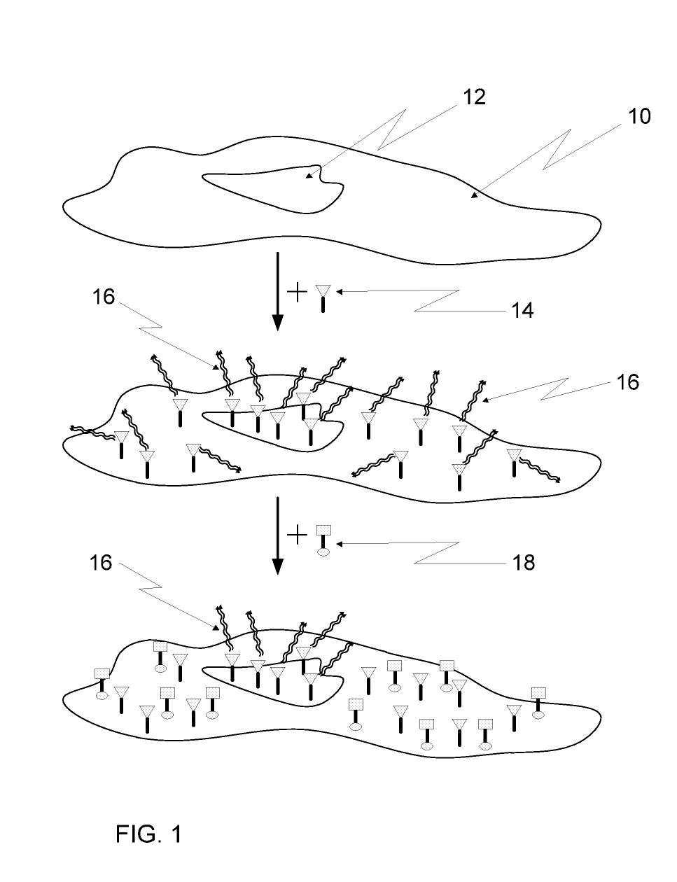

FIG. 1 depicts an embodiment illustrative for pairing 1 as described above, but similarly could also be understood as applicable to pairing 2. A first biological tissue 10 may be a normal, healthy, undiseased tissue. Alternatively, it could be a tissue in a first diseased state. The first tissue 10 may have a second tissue 12 present and growing within it. The second tissue 12 may be a diseased tissue such as a cancerous tumor growing within a healthy, undiseased tissue. Alternately, the second tissue 12 may be tissue in a second diseased state within the first tissue 10, which is in a first diseased state.

A first molecular entity 14 may be introduced to the tissues 10, 12. This first molecular entity 14 may provide some type of detectable signal 16. In one embodiment, this detectable signal 16 may be, for example, fluorescence which may be visually detectable by medical personnel. This first molecular entity 14 may be taken up by both of the first and second tissues 10, 12 so that it may be relatively evenly distributed throughout each of the first and second tissues. In this configuration, the entire mass of the first and second tissues 10, 12 may provide the detectable signal 16.

A second molecular entity 18 may be introduced to the first and second tissues 10, 12. The second molecular entity 18 may provide a blocking configuration that masks the detectable signal 16 that is produced by the first molecular entity 14. For example, in an embodiment in which the first molecular entity 14 fluoresces, the second molecular entity 18 may be configured as a fluorescent quencher to mask the fluorescence of the first entity. This second molecular entity 18 may be chosen or configured in such a way that it is taken up by one of the first tissue 10 or the second tissue 12 at a concentration that is greater than the concentration at which it is taken up by the other of the two tissues. A detectable characteristic difference may result between the two tissues 10, 12 such that the tissue having the greater concentration of the second molecular entity 18 evidences a correspondingly reduced amount of the detectable signal 16.

In one embodiment, the concentration difference from uptake of the second molecular entity 18 may be at least about 2 times greater for one of the tissues 10, 12. However, to provide better contrast, alternate embodiments may have a concentration difference of at least about 5 times greater, or at least about 10 times greater, or at least about 100 times greater. In one embodiment, the second molecular entity 18 may be taken up solely by only one of the two tissues 10, 12, thereby providing a contrast resolution for distinguishing between the two tissues. Such an embodiment is depicted in FIG. 1, wherein the second molecular entity 18 is taken up by substantially only the first tissue 10 and not by the second tissue 12. As such, only the second tissue 12 evidences the detectable signal 16 of the first molecular entity 14.

In alternate embodiments, the first molecular entity 14 and the second molecular entity 18 may be administered simultaneously, or the second molecular entity may be administered prior to the first molecular entity.

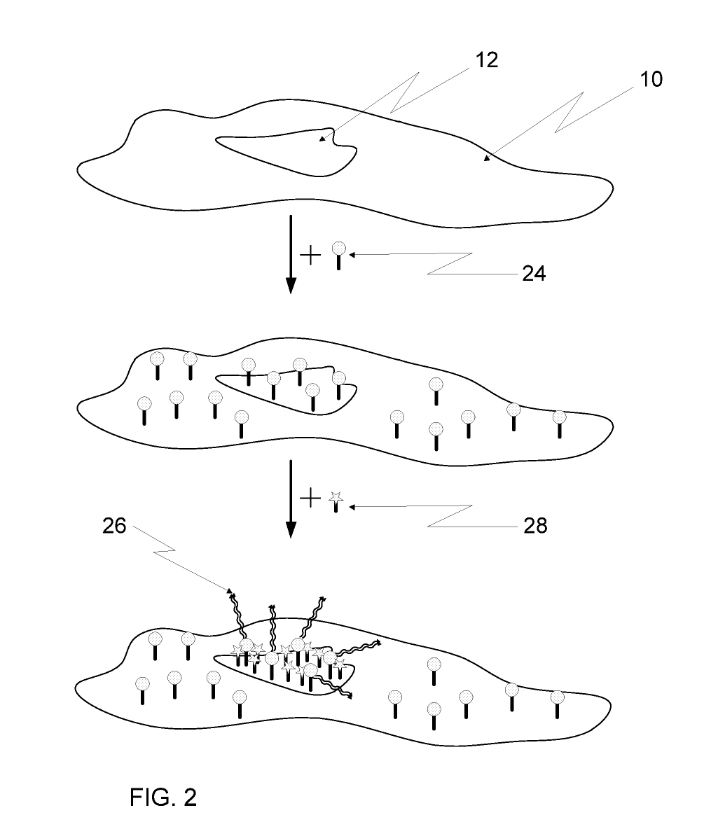

FIG. 2 depicts an embodiment illustrative for pairing 4 as described above, but similarly could also be understood as applicable to pairing 3. A first molecular entity 24 may be introduced to the tissues 10, 12. This first molecular entity 24 may be capable of providing a detectable signal 26 after it is activated. In one embodiment, this detectable signal 26 may be, for example, fluorescence which may be visually detectable by medical personnel. In various embodiments, this first molecular entity 24 may be taken up by either of the first tissue 10 or the second tissue 12, or as shown in FIG. 2, may be taken up by both of the first and second tissues so that it may be relatively evenly distributed throughout each of the first and second tissues. In this configuration, substantially the entire mass of the first and second tissues 10, 12 may have the first molecular entity 24 present, which entity is not yet producing its detectable signal 26.

A second molecular entity 28 may be introduced to the first and second tissues 10, 12. This second molecular entity 28 may provide an activating complex which in effect ‘turns-on’ the first molecular entity 24 so that the detectable signal 26 is produced. The second molecular entity 28 may be chosen or configured in such a way that it is taken up by one of the first tissue 10 or the second tissue 12 at a concentration that is greater than the concentration at which it is taken up by the other of the two tissues. A detectable characteristic difference may result between the two tissues 10, 12 such that the tissue having the greater concentration of the second molecular entity 28 evidences a correspondingly greater amount of the detectable signal 26.

In an embodiment, the concentration difference from uptake of the second molecular entity 28 may be at least about 2 times greater for one of the tissues 10, 12. However, to provide better contrast, alternate embodiments may have a concentration difference of at least about 5 times greater, or at least about 10 times greater, or at least about 100 times greater. In one embodiment, the second molecular entity 28 may be taken up solely by only one of the two tissues 10, 12, thereby providing a contrast resolution for distinguishing between the two tissues. Such an embodiment is depicted in FIG. 2, wherein the second molecular entity 28 is taken up by only the second tissue 12 and not by the first tissue 10. As such, only the second tissue 12 evidences the detectable signal 26 of the first molecular entity 24.

In alternate embodiments, the first molecular entity 24 and the second molecular entity 28 may be administered simultaneously, or the second molecular entity may be administered prior to the first molecular entity.

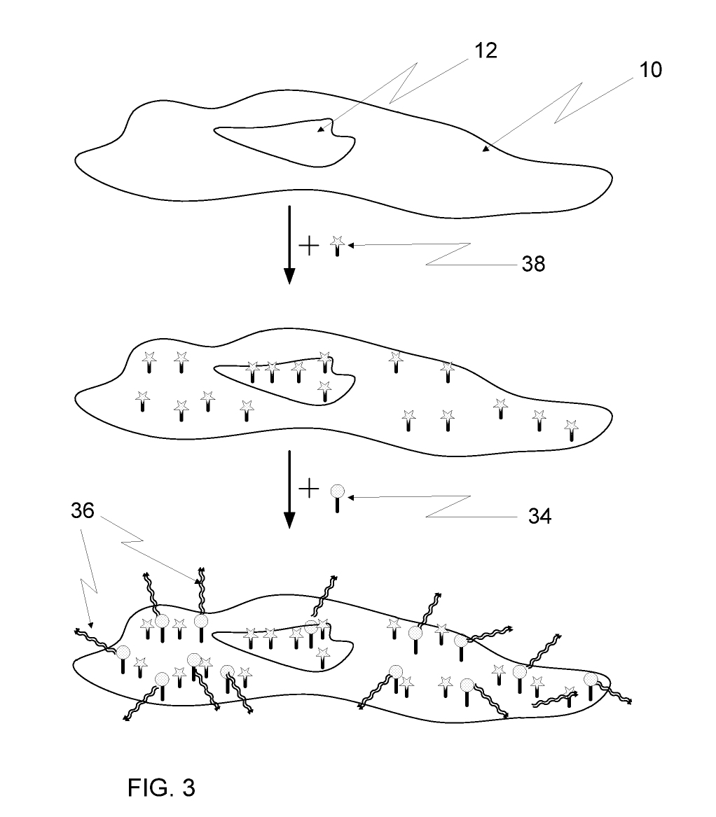

FIG. 3 depicts an embodiment illustrative for pairing 5 as described above, but similarly could also be understood as applicable to pairing 6. In this embodiment, the second, or activating entity 38 is introduced to the tissues 10, 12 prior to the introduction of the first or activatable entity 34. In the depicted embodiment, the activating entity 38 may be taken up by both of the first and second tissues 10, 12 so that it may be relatively evenly distributed throughout each of the first and second tissues. In this configuration, substantially the entire mass of the first and second tissues 10, 12 may have the activating entity 38 present.

The activatable entity 34 may be introduced to the first and second tissues 10, 12. This activatable entity 34 may be chosen or configured in such a way that it is taken up by one of the first tissue 10 or the second tissue 12 at a concentration that is greater than the concentration at which it is taken up by the other of the two tissues. A detectable characteristic difference may result between the two tissues 10, 12 such that the tissue having the greater concentration of the activatable entity 34 will produce a correspondingly greater amount of a detectable signal 36. As depicted in FIG. 3, the activatable entity 34 is taken up by the first tissue 10 at a concentration that is about 10 times greater than the concentration at which it is taken up by the second tissue 12. As a result, in this embodiment, the detectable signal 36 produced by the first tissue 10 may be about 10 times as intense as the detectable signal produced by the second tissue 12.

In an embodiment, a concentration difference from uptake of the activatable entity 34 may be at least about 2 times greater for one of the tissues 10, 12. However, to provide better contrast, alternate embodiments may have a concentration difference of at least about 5 times, at least about 10 times, or at least about 100 times. In an embodiment, the activatable entity 34 may be taken up solely by only one of the two tissues 10, 12, thereby providing a contrast resolution for distinguishing between the two tissues.

In alternate embodiments, the activatable entity 34 and the activating entity 38 may be administered simultaneously, or the activatable entity may be administered prior to the activating entity.

Several types of pairs of molecular entities may provide for the distinguishing of interspersed tissues using variations of the embodiments as discussed above with reference to the figures. One exemplary group of such entities may include pairs of fluorophores with fluorescence quenchers. Some examples of such pairs include:
doxorubicin with quencher suramin—doxorubicin (Dox) and suramin are both FDA approved chemotherapeutics (Dox for cancer and suramin for trypanosomiasis);
fluorescein with quenchers R-phycoerythrin, tryptophan or deoxyguanosine—fluorescein is an FDA approved fluorescent compound, that can be easily excitable and detectable; R-phycoerythrin (RPE) is a plant-derived (photosynthetic) pigment which is widely available, and tryptophan is an essential amino acid in humans and is available as a dietary supplement; and
chlorophyll with quencher quinones or xanthophylls—chlorophyll is the main photosynthetic pigment and the quencher molecules are widely available and easily synthesized.
   Some of these substances may already have specificity for binding to certain types of tissues while others may require structural or chemical modification to provide specificity.

One group of quenching compounds which may be usable in various embodiments is dark quenchers, or compounds which quench fluorescence by converting radiation to heat and thereby have no re-emittance of light. Some examples of dark quenchers include dabsyl (dimethylaminoazosulfonic acid), black hole quenchers, Qxl quenchers, IOWA BLACK® FQ, IOWA BLACK® RQ, and IRDYE® QC-1.

Antibody based pairs of fluorophores and quenchers may also be used in various embodiments. For the first fluorescing component, a fluorophore may be conjugated with an antibody having universal cell surface binding, and the quenching component may be conjugated with an antibody having a specific binding for healthy tissue. One such example may include anti-K5 with anti-K19, where K5 binds with normal mammary epithelial cells and K19 binds with diseased tissue.

Activation of the fluorophores, or causing them to fluoresce, may be brought about by illuminating them with a light source. The light source needed may be dependent on the fluorophore selected. In some embodiments, a light source spanning the visible spectrum may be used, or alternatively, a light source of about 450 nm to about 650 nm may be used. In some embodiments, a light source having a specific wavelength for excitation of the fluorophore may be used.

Some additional pairings of entities for use in visually distinguishing tissues may include pairings of entities in which one entity does not provide a detectable signal until it is coupled with, or in the presence of a second entity. Some examples of such pairings may include peroxide activated fluorescent probes or, in general, any fluorescible molecular probes, fluorescible molecular beacons and molecular hairpins, paired with their activating agent or agents.

Of the above-mentioned pairs, doxorubicin and suramin are already FDA approved, have been used in humans, and have a history of clinical safety. The pairing of doxorubicin and suramin would be useful in visualization of cancerous cells from non-cancerous cells, such as in liver tissue, for example. Doxorubicin has the structure as shown below and is a fluorophore which can be taken up non-preferentially by most tissues.

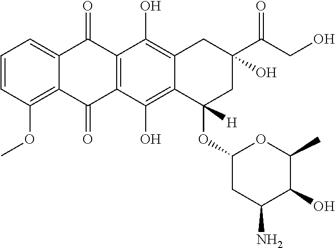 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

Doxorubicin is fluorescent and has at least one excitation at 546 nm with a broad emission that peaks at about 590 nm.

Suramin has the structure as shown below and has been found to quench the fluorescence of doxorubicin.

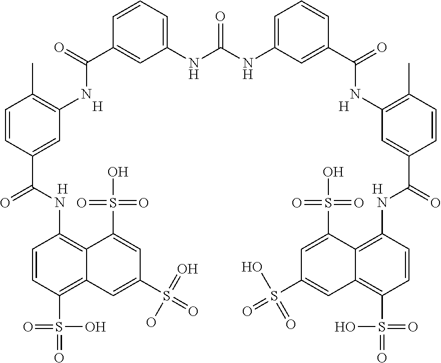

When applied in a similar concentration as doxorubicin, suramin may quench over 70% of the fluorescence of doxorubicin. Suramin, however, may lack the needed specificity for tissue binding to be used alone in conjunction with the doxorubicin for tissue visualization. Suramin may, however, be conjugated with an uptake agent such as superparamagnetic iron oxide particles (SPIOs) that may provide for binding of suramin with non-cancerous liver tissue. SPIO particles, and hence the suramin/SPIO conjugate, may typically be taken up only by healthy tissue, as nearly all liver tumors are deficient in Kupffer Cells and lack the ability to uptake SPIO particles.

Doxorubicin and suramin/SPIO conjugate may both be administered systemically, and concentration of the components in the liver may be facilitated by normal metabolism, which would typically tend to concentrate the molecules in the liver. Alternatively, the agents may also be administered locally by infusion directly to the liver tissue.

When used for liver resection, the doxorubicin and suramin/SPIO conjugate may be administered separately, or co-administered, prior to or at the time of the surgery. After a period of time sufficient for allowing the components to reach the liver tissue, illumination of the liver tissue during surgery with an appropriate excitation wavelength of light (546 nm) will cause the doxorubicin to fluoresce. The liver tumors will fluoresce from the doxorubicin while fluorescence of the surrounding liver tissue will be quenched by the suramin, thereby allowing for visualization of the tumor tissue via the naked eye or microscopy.

With approximately equal concentrations of doxorubicin and suramin at least about 70% of the fluorescence of the doxorubicin is quenched. This amount of quenching produces a contrast ratio of at least about 233% [calculated using the Weber ratio−change in intensity/intensity−(1−0.3)/0.3=233%]. This contrast ratio exceeds the level of discrimination of the human eye which is approximately 1-2%, thereby allowing for unaided visual discrimination between the cancerous and non-cancerous tissues.

Visualization may be supplemented or enhanced in various ways, some of which include, but are not limited to the use of microscopy (magnification), passive optical filters tuned to the emission wavelengths and possibly worn as glasses or goggles, active optical filters that amplify the emission wavelengths and possibly worn as glasses or goggles, active computer enhancement, or combinations thereof. Some examples of computer enhancement include, but are not limited to time averaging of exposures to increase signal-to-noise of a video feed, contrast variation, spectral filtering, photon multipliers, algorithmic enhancements, or combinations thereof.

Depending on the tissue of concern, binding agents may also be used to provide specificity for binding of the otherwise non-specific binding components which generally are taken up by many tissue types. As mentioned previously, doxorubicin is generally considered to be non-specific and binds to many different tissues. Such a compound could be rendered binding specific by conjugation with a binding agent. For liver specificity, lectins may be conjugated with the fluorophore or quencher since liver cells tend to express large numbers of lectin binding receptors. By using such binding agents, binding, distribution and/or concentration of otherwise non-specific binding entities may be improved in the tissues which are of concern.

In addition to using doxorubicin and suramin as discussed above, any derivatives of doxorubicin and suramin may also be usable in various embodiments for visually distinguishing two or more tissues.

Further, while the doxorubicin and suramin were discussed in the context of use for liver cancer, additional fluorophore/quenching pairs may be usable for visually distinguishing other cancers, diseased tissues, or diseased tissues having different stages, as well. In general, any pairing of compounds in which a detectable component, such as a fluorophore, is used along with a masking component, such as a fluorescing quencher, may be used in embodiments for distinguishing between two interspersed or adjacent biological tissues. As mentioned previously, if any of the components does not already have a desirable tissue binding specificity, such may be provided by conjugation with a carrier or uptake agent which does have binding specificity. Some examples of such carriers or uptake agents may include, but are not limited to, superparamagnetic iron oxide particles, antibodies, biologics, and combinations thereof. Various compounds may be used for conjugation of the fluorescing component or quenching component with the carrier or uptake agent. Some examples of conjugating substances may include, but are not limited to, polyethyleneimine, polyethylene glycol, bovine serum albumin, biotin-avidin complexes, and combinations thereof.

### Example

### Liver Tumor Resection

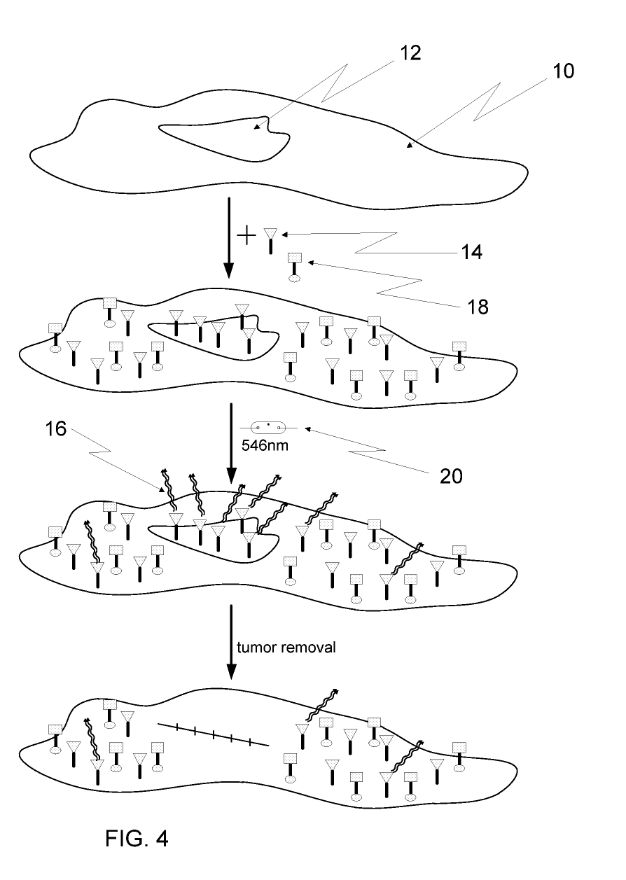

FIG. 4 depicts an illustration of an embodiment for a liver tumor resection. The cancerous tumor 12 will be resected from the non-cancerous liver tissue 10 with the use of doxorubicin 14 as the fluorescing agent and suramin/SPIO conjugate 18 as the fluorescent quencher. Prior to the surgery, PET images of the tumor will be taken and provided for the surgeon to use as a general guide for location of the tumor. The doxorubicin 14 and the suramin/SPIO conjugate 18 will be administered prior to the surgery to make it easier for the surgeon to distinguish between the cancerous tissue 12 and non-cancerous liver tissue 10. Dosing requirements for the doxorubicin 14 and the suramin/SPIO conjugate 18 may be determined with consideration that sufficient fluorescence be present for visualization and sufficient quenching be present to provide contrast between the cancerous tissue 12 and non-cancerous tissue 10. In clinical settings, the dosing, dose amounts, and other parameters will be determined as a function of specific parameters. Some of these dosing parameters may include patient history, patient characteristics, previous chemotherapy, and whether the components will be infused directly to the liver or systemically.

For this example, the doxorubicin 14 will be dosed similarly as may be done for chemotherapy, which is about 60-75 mg/m² (milligrams per square meter) body area, and given as a single IV injection over 15 minutes to provide a goal of about 1 μM (micromolar) to about 50 μM (micromolar) concentration in the liver. Doxorubicin 14 has a distributive half-life of approximately 5 minutes and a terminal half-life of 20 to 48 hours. For a human body of approximately 82 kg (kilograms) and 1.73 m (meters) (5′8″) in height, the approximate body surface area is about 2 m² (square meters). The above dosing will be able to provide a concentration of about 594 μM (micromolar) of doxorubicin 14 in the body, and given the terminal half-life and typical known clearance through the liver, this can achieve the stated concentration goals in the liver.

The SPIO/suramin conjugate 18 will be co-administered with the doxorubicin 14 and dosed similarly as may be done for dosing non-modified SPIOs for imaging, which is about 15 μM/kg (micromol per kilogram) of body weight, diluted in 100 ml (milliliters) of 5% glucose and delivered over 30 minutes, with a goal of a concentration in the liver of at least the same level as the doxorubicin 14. Providing similar concentrations of suramin and doxorubicin in tissue has been shown to quench doxorubicin fluorescence by over 70%, which will provide a contrast sufficient for distinguishing the cancerous liver tissue from the non-cancerous liver tissue.

About an hour after dosing, the patient will be presented for the resection. A light source 20 of about 546 nm (green) will be provided in the vicinity of the surgery to illuminate the liver. The surgeon will cut into the liver at a location determined by the PET images, and when the tumor is exposed, the light source will cause the tumor to fluoresce 16 with the emission of a broad peak near 590 nm (yellow-orange). This emission will be visible to the naked eye, and the surgeon will be able to remove cancerous tissue 12.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method for distinguishing interspersed first and second biological tissues, the method comprising administering an effective amount of each of a first component and a second component to the interspersed tissues, wherein:
the first component is detectable and taken-up by both of the first and second biological tissues; and
the second component masks detectability of the first component and is taken-up by one of the first and second biological tissues at a concentration which is greater than a concentration which is taken up by the other of the first and second biological tissues to mask detectability of the first component in the one of the first and second biological tissues to a greater extent than any masking of detectability of the first component in the other of the first and second biological tissues.

2. The method of claim 1, wherein the concentration of the second component in the one of the first and second tissues is at least about 1.1 times greater than the concentration of the second component in the other of the first and second tissues.

3. (canceled)

4. The method of claim 1, wherein the first biological tissue comprises healthy biological tissue and the second biological tissue comprises diseased biological tissue, and the method further comprises:
selecting as the first component, a component which is taken-up by both of the healthy biological tissue and the diseased biological tissue; and
selecting as the second component, a component which is taken-up by substantially only one of the healthy biological tissue and the diseased biological tissue.

5. The method of claim 1, wherein the first biological tissue comprises biological tissue having a first diseased state, and the second biological tissue comprises biological tissue having a second diseased state, and the method further comprises:
selecting as the first component, a component which is taken-up by both of the biological tissue having the first diseased state and the biological tissue having the second diseased state; and
selecting as the second component, a component which is taken-up by one of the biological tissue having the first diseased state and the biological tissue having the second diseased state.

6.-7. (canceled)

8. The method of claim 1, wherein the first biological tissue comprises non-cancerous liver tissue and the second biological tissue comprises cancerous liver tissue, and the method further comprises:
selecting as the first component, a component which is taken-up by both of the non-cancerous liver tissue and the cancerous liver tissue; and
selecting as the second component, a component which is taken-up by substantially only the non-cancerous liver tissue.

9. The method of claim 1, wherein the first component comprises a fluorophore and the second component comprises a masking component which quenches fluorescence of the fluorophore.

10. The method of claim 9, wherein the second component comprises a dark quencher.

11. The method of claim 1, wherein the first biological tissue comprises non-cancerous tissue and the second biological tissue comprises cancerous tissue, and the method further comprises:
selecting as the first component, a fluorophore which is taken-up by both of the non-cancerous biological tissue and the cancerous biological tissue; and
selecting as the second component, a masking component which quenches fluorescence of the fluorophore and which is taken-up by substantially only the cancerous biological tissue.

12. The method of claim 11, wherein the fluorophore and masking component comprise a pairing of: doxorubicin and suramin; fluorescein and R-phycoerythrin; fluorescein and tryptophan; fluorescein and deoxyguanosine; chlorophyll and quinones; chlorophyll and xanthophylls; antibodies anti-K5 and anti-K19, and one of anti-K5 and anti-K19 is conjugated with a fluorophore and the other of anti-K5 ad anti-K19 is conjugated with a fluorescence quencher; or an antibody conjugated fluorophore having universal cell surface binding and an antibody conjugated quencher with specific binding for non-cancerous tissue; or any combination thereof.

13. (canceled)

14. The method of claim 1, wherein the first biological tissue comprises non-cancerous liver tissue and the second biological tissue comprises cancerous liver tissue, and the method further comprises:
selecting doxorubicin as the first component, doxorubicin being taken-up by both of the non-cancerous liver tissue and the cancerous liver tissue; and
selecting suramin as the second component, suramin being taken-up by only the non-cancerous liver tissue.

15. The method of claim 14, wherein the suramin is conjugated with an uptake agent specific for binding with non-cancerous liver tissue.

16. The method of claim 15, wherein the uptake agent is superparamagnetic iron oxide particles, an antibody, or a biologic, or any combination thereof.

17. The method of claim 14, wherein the suramin is conjugated with superparamagnetic iron oxide particles by polyethyleneimine, polyethylene glycol, bovine serum albumin, or biotin-avidin complexes, or any combination thereof.

18. (canceled)

19. The method of claim 1, wherein the first biological tissue comprises non-cancerous liver tissue and the second biological tissue comprises cancerous liver tissue, and the method further comprises:
selecting doxorubicin as the first component, doxorubicin being taken-up by both of the non-cancerous liver tissue and the cancerous liver tissue;
selecting suramin as the second component, suramin being taken-up by only the non-cancerous liver tissue; and
illuminating the non-cancerous liver tissue and cancerous liver tissue with a light source to fluoresce the doxorubicin in the cancerous liver tissue.

20. The method of claim 19, wherein:
the illuminating of the non-cancerous liver tissue and cancerous liver tissue comprises illuminating with a light source having at least one wavelength within a range of wavelengths from about 450 nm to about 650 nm; and
the method further comprises enhancing an appearance of the fluorescing doxorubicin in the cancerous liver tissue by magnification, optical filtering, computer enhancement with contrast variation, computer enhancement with time-averaging, computer enhancement with spectral filtering, computer enhancement with algorithm applications, or combinations thereof.

21.-22. (canceled)

23. A method for distinguishing interspersed first and second biological tissues, the method comprising administering an effective amount of each of a first component and a second component to the interspersed tissues, wherein:
the first component is capable of being detectable upon activation by the second component;
one of the first and second components is taken-up by both of the first and second biological tissues; and
the other of the first and second components is taken-up by only one of the first and second biological tissues.

24. The method of claim 23, wherein one of:
the first biological tissue comprises un-diseased biological tissue and the second biological tissue comprises diseased biological tissue; and
the first biological tissue comprises non-cancerous biological tissue and the second biological tissue comprises cancerous biological tissue.

25. (canceled)

26. The method of claim 23, wherein the first component comprises a fluorescent probe and the second component comprises peroxide for activation of the fluorescent probe.

27. The method of claim 23, wherein the first component comprises one of a fluorescible molecular probe or a fluorescible molecular beacon; and the second component provides an activating stimulus for activating fluorescence of the molecular probe or molecular beacon.

28. A method for resection of tissue to remove cancerous tissue from non-cancerous tissue, the method comprising:
selecting a first component and a second component, wherein:
the first component is detectable and is taken-up by both the non-cancerous tissue and the cancerous tissue, and
the second component masks detectability of the first component and is taken-up by one of the non-cancerous tissue and the cancerous tissue at a concentration which is greater than a concentration which is taken up by the other of the non-cancerous tissue and the cancerous tissue to enable the non-cancerous tissue and the cancerous tissue to be distinguishable from one another;
administering to a patient an effective amount of each of the first component and the second component; and
surgically removing the distinguishable cancerous tissue from the patient.

29.-30. (canceled)

31. The method of claim 28, wherein:
the first component comprises a fluorophore;
the second component comprises a masking component which quenches fluorescence of the fluorophore; and
the method further comprises illuminating the cancerous tissue and the non-cancerous tissue with a light source to fluoresce the first component.

32. The method of claim 31, wherein the fluorophore and masking component comprise a pairing of: doxorubicin and suramin; fluorescein and R-phycoerythrin; fluorescein and tryptophan; fluorescein and deoxyguanosine; chlorophyll and quinones; chlorophyll and xanthophylls; an antibody conjugated fluorophore having universal cell surface binding and an antibody conjugated quencher with specific binding for non-cancerous tissue; or combinations thereof.

33. (canceled)

34. The method of claim 28, wherein the tissue comprises liver tissue.

35. The method of claim 28, wherein the administering comprises at least one of administering the effective amount of the first component simultaneously with the administering of the effective amount of the second component, and administering the effective amount of the first component and subsequently administering the effective amount of the second component.

36. (canceled)

37. The method of claim 28, wherein the administering comprises at least one of administering the effective amount of each of the first component and the second component systemically into the patient by means of an intravenous device, and administering the effective amount of each of the first component and the second component directly to the tissue by infusion.

38.-39. (canceled)

40. The method of claim 28, wherein:
the tissue comprises liver tissue;
the first component comprises at least one of doxorubicin and derivatives thereof; and
the second component is taken-up by only the non-cancerous liver tissue and comprises at least one of suramin and derivatives thereof.

41. The method of claim 28, wherein:
the tissue comprises liver tissue;
the first component comprises doxorubicin;
the second component comprises suramin conjugated with superparamagnetic iron oxide particles to inhibit uptake of the suramin by the cancerous liver tissue;
the doxorubicin is capable of being fluoresced with a light source to fluoresce the cancerous liver tissue; and
the administering of the second component comprises administering the suramin-superparamagnetic iron oxide conjugate.

42. The method of claim 41, wherein the administering an effective dose comprises administering sufficient doxorubicin and suramin-iron oxide conjugate to provide a suramin-iron oxide conjugate concentration in the liver which is at least substantially the same as a doxorubicin concentration in the liver.

43. The method of claim 42, wherein the concentration of doxorubicin in the liver is from about 1 micromolar to about 50 micromolar and the concentration of suramin-iron oxide conjugate in the liver is from about 1 micromolar to about 100 micromolar.

44.-47. (canceled)
